(19) United States
(12) Patent Application Publication
(10) Pub. No.: US 2021/0386464 A1
(43) Pub. Date: Dec. 16, 2021

(54) INTERNAL FIXATOR APPARATUS FOR DISTRACTION OSTEOGENESIS

(71) Applicants: POLYVALOR, LIMITED PARTNERSHIP, Montreal (CA); SHRINERS HOSPITALS FOR CHILDREN, Montreal (CA)

(72) Inventors: Jérémie GAUDREAU, Ottawa (CA); Isabelle VILLEMURE, Boucherville (CA); Mina MEKHAIL, Montreal (CA); Reggie HAMDY, Verdun (CA)

(21) Appl. No.: 17/279,631

(22) PCT Filed: Oct. 4, 2019
(86) PCT No.: PCT/CA2019/051426

§ 371 Date: Mar. 25, 2021

Related U.S. Application Data
(60) Provisional application No. 62/741,139, filed on Oct. 4, 2018.

(51) Int. Cl. A61B 17/80

(52) U.S. Cl. CPC A61B 17/8019; A61B 2017/00876

(57) ABSTRACT

An internal fixator apparatus comprises a barrel member having a bone interface adapted to be anchored to a first part of a bone in an extramedullary connection. A piston member has a bone interface adapted to be anchored to a first part of a bone, the piston member including a threaded nut portion, the barrel member and the piston member being operatively connected to concurrently form a joint whereby the barrel member and the piston member are displaceable at least in translation relative to one another. A fixator mechanism is inside the barrel member and the piston member, the fixator mechanism comprising at least a leadscrew threadingly engaged with the threaded nut portion, and at least one magnet connected to the leadscrew to rotate concurrently therewith, the magnet being rotatingly received in the barrel member.

### TECHNICAL FIELD

The present disclosure relates to an internal fixator apparatus used to perform distraction osteogenesis.

### BACKGROUND OF THE ART

Distraction osteogenesis (DO) is a surgical technique that has been used for decades to lengthen long bones. This allows for treatment of limb length discrepancies (LLD), limb deformities and other related illnesses. DO is used in both adults and children. However since children have not reached their full developed bones they need specific devices in order to preserve their bone growth capacity. Currently used techniques involve the application of an external fixator on the affected bone, followed by an osteotomy (i.e. a cut), and gradual distraction of the two bone segments. This controlled distraction generates new bone within the distracted gap. When the bone has been sufficiently lengthened, the gradual distraction of the gap is stopped, and the bone is left to consolidate. In children, this technique is executed by applying an external fixator to the targeted bone and manually distracting the apparatus over a course of a few months.

A well-known external fixator is called the llizarov apparatus. The llizarov apparatus is a bulky external fixator in children may lead to numerous social, psychological and medical complications, such as social isolation due to body image, anxiety, and pin-site infection. Compliance to the distraction procedure is another issue, since the children or their parents may have to perform the distraction manually a few times or several times a day. Moreover, since it is a manual distraction, there is possible human error involved.

Internal fixators for bone elongation are also known, such as intramedullary nails that distract a bone using a magnetic remote control. However, intramedullary nail geometry may interfere with growth plates of long bones, and this may affect normal physiological bone development in growing children. Moreover, intramedullary nails are relatively expensive, with documented cases of mechanical failure or jam in patients. Therefore, there are currently limited alternatives on the market for internal plate fixators designed with an integrated bone-accelerating technology to improve patient care and reduce treatment time, and no alternatives for an internal fixator that does not interfere with the patient's growth plates.

### SUMMARY

It is an aim of the present disclosure to provide an internal fixator that addresses issues related to the prior art.

In accordance with the present disclosure, there is provided an internal fixator apparatus comprising: a barrel member having a bone interface adapted to be anchored to a first part of a bone in an extramedullary connection, a piston member having a bone interface adapted to be anchored to a first part of a bone, the piston member including a threaded nut portion, the barrel member and the piston member being operatively connected to concurrently form a joint whereby the barrel member and the piston member are displaceable at least in translation relative to one another, and a fixator mechanism inside the barrel member and the piston member, the fixator mechanism comprising at least a leadscrew threadingly engaged with the threaded nut portion, and at least one magnet connected to the leadscrew to rotate concurrently therewith, the magnet being rotatingly received in the barrel member.

Further in accordance with the present disclosure, as an example, the magnet is a permanent magnet received in a housing.

Still further in accordance with the present disclosure, as an example, the housing has shaft portions.

Still further in accordance with the present disclosure, as an example, one of the shaft portions is rotatably connected to the barrel member by a bearing.

Still further in accordance with the present disclosure, as an example, the bearing is supported by an end cap of the barrel member, the end cap plugging an end of a tube of the barrel member.

Still further in accordance with the present disclosure, as an example, the housing is coupled to a remainder of the fixator mechanism by one of the shaft portions.

Still further in accordance with the present disclosure, as an example, the fixator mechanism has a reduction mechanism reducing a speed of rotation from the magnet to the leadscrew.

Still further in accordance with the present disclosure, as an example, the barrel member has a tube portion slidingly received in an annular gap of the piston member.

Still further in accordance with the present disclosure, as an example, the barrel member has at least a first tube and a second tube connected to one another and concurrently defining an inner cavity of the barrel member, the tube portion slidingly received in the annular gap of the piston member being part of the second tube.

Still further in accordance with the present disclosure, as an example, an anti-rotation coupling is defined between the tube portion and the piston member.

Still further in accordance with the present disclosure, as an example, the first tube has an internal flange.

Still further in accordance with the present disclosure, as an example, a bearing is supported by the internal flange, the bearing being rotatably connected to the fixator mechanism.

Still further in accordance with the present disclosure, as an example, a third tube may be in the barrel member, the first tube and the third tube forming another annular gap in which the second tube is received, the second tube projecting out of the other annular gap to define the tube portion cooperating with the piston member.

Still further in accordance with the present disclosure, as an example, the third tube has an internal flange, a bearing being supported by the internal flange, the bearing being rotatably connected to the fixator mechanism.

Still further in accordance with the present disclosure, as an example, the piston member has a first tube and a second tube connected to one another and concurrently defining an inner cavity of the piston member including the threaded nut portion, the first tube and a second tube of the piston member defining the annular gap of the piston member.

Still further in accordance with the present disclosure, as an example, the fixator mechanism includes a flexible coupling between the leadscrew and a remainder of the fixator mechanism.

Still further in accordance with the present disclosure, as an example, the barrel member has a tubular body with a diameter ranging between 12 and 20 mm.

Still further in accordance with the present disclosure, as an example, the bone interface of the barrel member and/or of the piston member is a plate projecting laterally from a tubular body of the barrel member and/or of the piston member.

Still further in accordance with the present disclosure, as an example, piston member and the barrel member both have the plate as the bone interface.

In accordance with a further embodiment of the present disclosure, there is provided a system comprising: the internal fixator apparatus described above, and a fixator actuator including at least one rotating magnet.

### DESCRIPTION OF THE DRAWINGS

FIG. 1 is a perspective view of an internal fixator apparatus in accordance with the present disclosure, relative to a bone and prior to expansion or elongation in distraction osteogenesis;

FIG. 2 is a perspective view of the distracted internal fixator apparatus of FIG. 1, with osteotomy and distraction on the bone;

FIG. 3 are perspective views of the internal fixator apparatus of FIG. 1, showing also a fixator mechanism as assembled and as exploded;

FIG. 4 is a longitudinal cross-section view of the internal fixator apparatus of FIG. 1;

FIG. 5 is a schematic view of a magnetic drive in an internal fixator system in accordance with the present disclosure, with a) initial position; b) eighth of a turn; c) quarter turn;

FIG. 6 is a longitudinal cross-section view of another embodiment of the internal fixator apparatus of FIG. 1; and

FIG. 7 are perspective views of the internal fixator apparatus of FIG. 6, showing also a fixator mechanism as assembled and as exploded.

### DETAILED DESCRIPTION

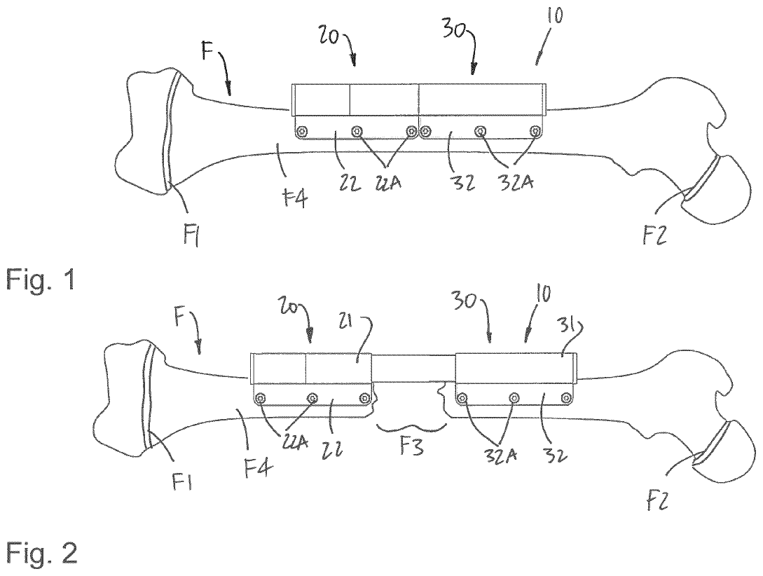

Referring to the drawings and more particularly to FIGS. 1 and 2, there is illustrated an internal fixator apparatus 10 in accordance with the present disclosure, as mounted to a femur F, in extramedullary connection (i.e., on the surface of the bone, and not in intramedullary connection). While shown as being mounted to the femur F, the internal fixator apparatus 10 may be used with other bones, such as long bones like the tibia, the fibula, the humerus, the radius, the ulna. The internal fixator apparatus 10 is mounted to the shaft of the femur, between the physis F1 and F2 of the femur F (i.e., growth plates). Also shown in FIGS. 1 and 2 is a gap F3 resulting from osteotomy and distraction, with the internal fixator apparatus 10 anchored to opposite sides of the gap F3, on the diaphysis F4, for instance as a result of distraction osteogenesis (DO).

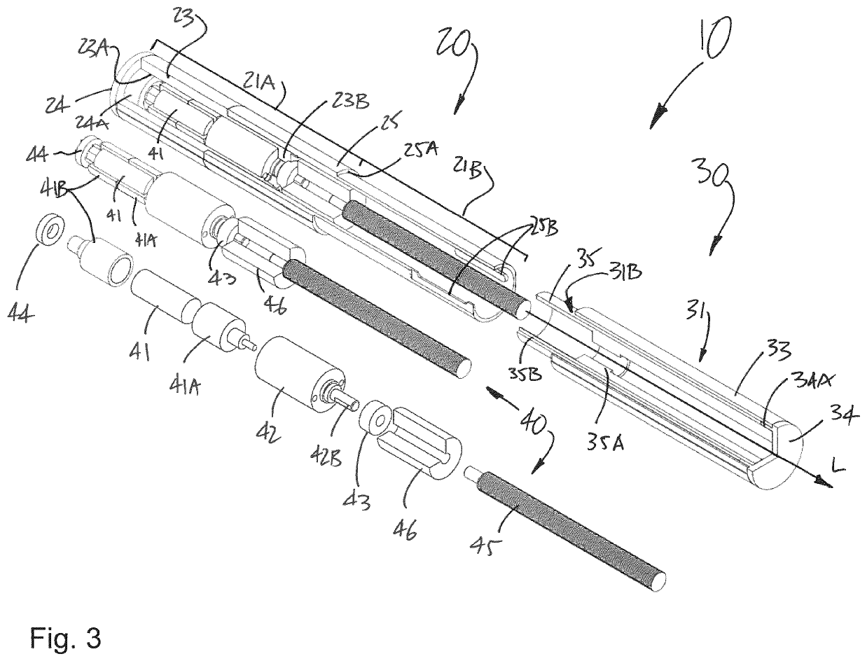

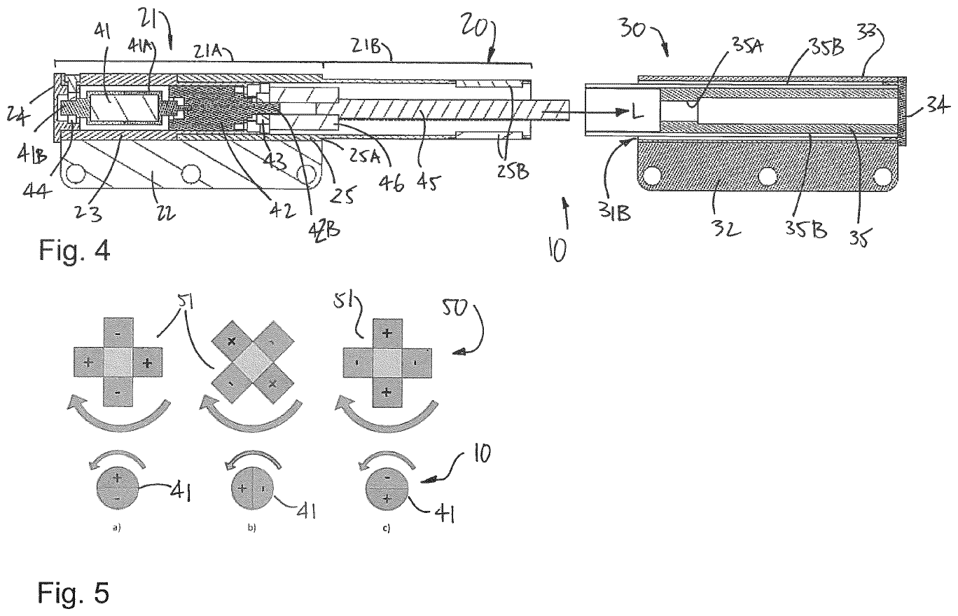

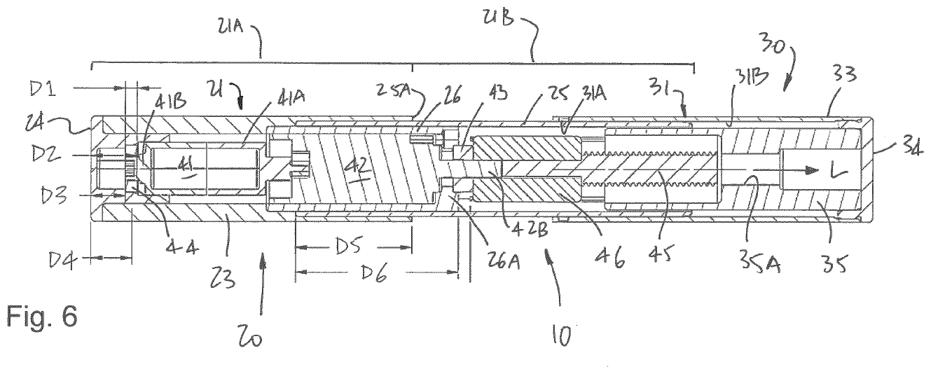

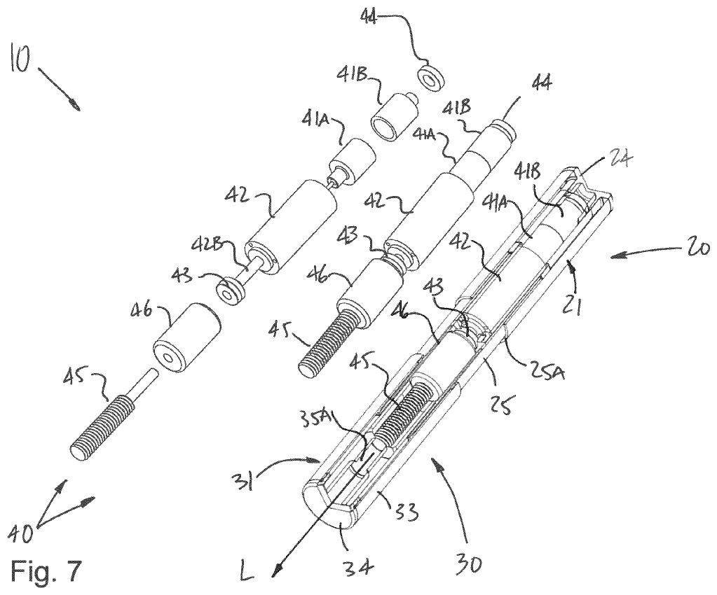

Referring to FIGS. 1 to 4 and to FIGS. 6 and 7, the internal fixator apparatus 10 has a barrel member 20, a piston member 30, and a fixator mechanism 40, in two embodiments. FIGS. 3 and 4 show the interior of a first embodiment of the internal fixator apparatus 10, whereas FIG. 6 shows the interior of a second embodiment of the internal fixator apparatus 10. As the embodiments share numerous components, like reference numerals will be used herein between embodiments. A fixator actuator 50 may also be provided to control the length of the internal fixator apparatus 10, and actuate an expansion or contraction of the internal fixator apparatus 10. In an embodiment the internal fixator apparatus 10 is passive (i.e., not powered by an electrical signal) as it is operated during DO by being exposed to a given magnetic field, wherein the fixator actuator 50 can control the expansion or contraction of the internal fixator apparatus 10 remotely. It is however contemplated to provide a motorization unit and power source in the internal fixator apparatus 10.

Referring to FIGS. 1 to 4, the barrel member 20 is shown as having a tubular body 21 from which projects a bone interface 22. As in FIGS. 1 and 2, the bone interface 22 may be in the form of a fixing plate, by which the barrel member 20 is anchored extramedullarily to the bone F by way of fasteners 22A (e.g., locking screws, nails, etc). Other bone interface configurations are contemplated as an alternative to a fixing plate, such as brackets, collars, etc.

The tubular body 21 of the barrel member 20 may have different portions, such as a structural casing portion 21A and a joint portion 21B. The structural casing portion 21A is the portion of the tubular body 21 that supports the bone interface 22, and that accommodates some of the immovable components of the fixator mechanism 40. The joint portion 21B on the other hand may collaborate with the piston member 30 to guide the translational movement of the piston member 30 relative to the barrel member 20. The joint portion 21B may enclose the rotatable component of the fixator mechanism 40 as detailed hereinafter. In the embodiment of FIGS. 6 and 7, the bone interface 22 is not visible due to the location of the point of view, but the bone interface 22 may be present and may project from the barrel member 20.

Referring to FIGS. 3 and 4, an exemplary construction of the barrel member 20 is shown, with a first tube 23 having an end cap 24 at a first end, and with a second end of the first tube 23 being open ended. The first tube 23 and the end cap 24 are shown as being separate components, as the two-part assembly of the first tube 23 and end cap 24 of FIGS. 3 and 4 may be simpler to fabricate and may facilitate the insertion of components in an inner cavity 23A of the first tube 23. A bearing support 23B may be provided adjacent to or at the second end of the first tube 23. The bearing support 23B may for instance be in the form an internally projecting flange with central bore, but could also be an annular channel(s) or seat, etc. Circlips could also be used as bearing support 23B. The first tube 23 may also have a constant inner diameter without any add-on features. The end cap 24 may have a tube member 24A configured to be received in the inner cavity 23A of the first tube 23, The tube member 24A, if present, may enclose some of the components of the fixator mechanism 40. In an embodiment, the tube member 24A may be force-fitted to into the inner cavity 23A of the first tube 23. As shown in FIG. 4, a fastener(s) such as a set screw may be used to secure the end cap 24 to the first tube 23.

The barrel member 20 may also have a second tube 25, with both ends of the second tube 25 being open. The second tube 25 may have an outer diameter being the same as the outer diameter of the first tube 23 such that, when assembled end to end, the tubes 23 and 25 form a continuously smooth surface. A shoulder 25A may be formed on the outer surface of the second tube 25, at a reduction of outer diameter of the second tube 25. In an inner cavity of the second tube 25, one or more blocks 25B may be present. The second tube 25 may be welded/bonded to the first tube 23 after insertion and attachment of components therein. Referring to FIGS. 6 and 7, in another embodiment, the second tube 25 is of smaller diameter than the first tube 23. Accordingly, in the embodiment of FIGS. 6 and 7, the shoulder 25A is defined by the reduction of diameter from the first tube 23 to the second tube 25. In the embodiment of FIGS. 6 and 7, the second tube 25 may be welded/bonded to the first tube 23 after insertion and attachment of components therein. Still in the embodiment of FIGS. 6 and 7, another tube, shown as tube 26, may include a bearing support 26A, as an alternative to the bearing support 23B of the embodiment of FIGS. 3 and 4. The tubes 23 and 26 may define a continuously smooth inner surface of the inner cavity 23A, though this is optional. An annular gap between the tubes 23 and 26 may serve to accommodate an end of the second tube 25, in the manner shown in FIGS. 6 and 7. In an embodiment, the tube 26 is integral with the gearbox 42 described below.

Accordingly, as shown in FIGS. 3 and 4, the barrel member 20 may be constituted of three components, namely the first tube 23, the end cap 24 and the second tube 25, or it may be constituted of four components, as in FIGS. 6 and 7, namely the first tube 23, the end cap 24, the second tube 25 and tube 26, that may be referred to as a third tube, for reference purposes. It is also contemplated to use a barrel member 20 that is made of a single monolithic part or of two parts. For example, if a bearing support is present, such as the bearing support 23B, it may be part of the second tube 25. In such a case, the first tube 23 could be without the end cap 24. The structural casing portion 21A of the tubular body 21 may be formed of the first tube 23, of the end cap 24 (if present) and of the larger outer diameter segment of the second tube 25 (FIGS. 3 and 4), or of the first tube 23 alone or with the end cap 24 if present, as in FIG. 6. The joint portion 21B of the tubular body 21 may be formed of the smaller outer diameter segment of the second tube 25. It is contemplated to use additive manufacturing techniques, such as 3D printing, stereolithography, etc, to make the barrel member 20 in a monolithic configuration. Electro-erosion may also be used.

Referring to FIGS. 1 to 4, the piston member 30 is shown as having a tubular body 31 from which projects a bone interface 32. As in FIGS. 1 and 2, and in similar fashion to the bone interface 22 of the barrel member 20, the bone interface 32 may be in the form of a fixing plate, by which the piston member 30 is anchored extramedullarily to the bone F by way of fasteners 32A (e.g., screws, nails, etc). The fasteners 32A may be locking screws, or like fasteners, that maintain a constant gap between the bone and the bone interfaces 22,32, so as not to impede surface vascularisation on the bone. Other bone interface configurations are contemplated as an alternative to a fixing plate, such as brackets, collars, etc. In the embodiment of FIGS. 6 and 7, the bone interface 32 is not visible due to the location of the point of view, but the bone interface 32 may be present and may project from the piston member 30.

Referring to FIGS. 3 and 4, an exemplary construction of the piston member 30 is shown, with a first tube 33 forming the exposed surface of the piston member 30. The first tube 33 may have a constant inner diameter without any add-on features. An end cap 34 may be at a first end of the first tube 33, with a second end of the first tube 33 being open ended. The first tube 33 the end cap 34 are shown as being separate components, as the two-part configuration of FIGS. 3 and 4 may be simpler to fabricate. The end cap 34 may have a tube member 34A configured to facilitate the assembly of the first tube 33 with a second tube 35.

The piston member 30 may also have the second tube 35, with both ends of the second tube 35 being open. The second tube 35 may have nut portion 35A having internal threading. The nut portion 35A may be in a narrowing portion of the second tube 35 as in FIGS. 3 and 4. The nut portion 35A may be integrally monolithic with a remainder of the second tube 35, or may be an add on part that would be received and anchored in an inner cavity of the second tube 35. In the illustrated embodiment, the internal threads are made directly into the material of the nut portion 35A.

The second tube 35 has an outer diameter being smaller than the inner diameter of the first tube 33 such that, when assembled concentrically as in FIGS. 3 and 4, the tubes 33 and 35 form an annular cavity 31B therebetween, for matingly receiving therein the joint portion 21B of the barrel member 20. The second tube 35 may be longer than the first tube 33, to increase a contact surface between the barrel member 20 and the piston member 30, to enhance a structural integrity of the internal fixator apparatus 10 and provide it with a high flexural rigidity. One or more straight grooves 35B (a.k.a., splines) may be defined on an outer surface of the second tube 35, for collaborating with the blocks 25B in the barrel member 20. The collaboration between the blocks 25B and the straight grooves 35B constrain the movement of the piston member 30 relative to the barrel member 20 to a translation along distraction direction L, as the blocks 25B and grooves 35B block any substantial rotation between the barrel member 20 and the piston member 30. As an alternative to the arrangement shown, one or more grooves could be on the barrel member 20 with corresponding block(s) on the piston member 30. In the embodiment of FIGS. 6 and 7, the second tube 35 may be shorter than the first tube 33. This arrangement may also be used in the embodiment of FIGS. 3 and 4. Though not shown, anti-rotation features such as the blocks 25B and grooves 35B may be present in the embodiment of FIGS. 6 and 7.

Accordingly, as shown in FIGS. 3 and 4, the piston member 30 may be constituted of three components, namely the first tube 33, the end cap 34 and the second tube 35. It is also contemplated to use a piston member 30 that is made of a single monolithic part or of two parts. For example, additive manufacturing techniques, such as 3D printing, stereolithography, etc, may be used to make the piston member 30 in a monolithic configuration. The barrel member 20 and the piston member 30 are assembled in the manner shown in FIGS. 1 and 2, such that they may move along the elongated direction of the internal fixator apparatus 10, i.e., distraction direction L. The barrel member 20 and the piston member 30 may be fabricated with tight tolerances to ensure a precise close proximity fit when the joint portion 21B of the barrel member 20 is received in the annular cavity 31B, with the assembly constrained to strict translational degree of freedom expansion/contraction. The resulting assembly may form a barrier against bodily fluid infiltration, essentially shielding the fixator mechanism 40 from the bodily fluids. It is also contemplated to use a seal, such as a seal made of medical-grade rubber, silicone, etc, for instance received in an annular channel 31A (FIG. 6, also possibly present in the embodiment of FIGS. 3 and 4). Because of their internal use, the barrel member 20 and the piston member 30 are made of medical grade materials, such as titanium or stainless steel. As the internal fixator apparatus 10 may be subjected to the high forces and pressures related to DO, the use of metallic materials is well suited though high rigidity polymers could be contemplated as well.

Referring to FIGS. 3 and 4 and/or to FIGS. 6 and 7, the fixator mechanism 40 may have one or more magnets 41 (one in the embodiment shown) to operate a DO process using for example a magnetic field process. The magnet 41 may be a permanent magnet(s) that may be accommodated in a housing including housing members 41A and 41B, with appropriate shaft members to couple the magnet 41 to other components of the fixator mechanism 40. In an embodiment, the magnet 41 is separated in a North half, and a South half, a separation between the polarities being for example a plane incorporating direction L. According to an embodiment, a gearbox 42 is coupled to the magnet 41 by way of a shaft portion on the housing member 41A. However, the fixator mechanism 40 may be without the gearbox 42, with the magnet 41 connected directly to the leadscrew 45. The gearbox 42 is for instance a reduction gearbox or any other type of reduction mechanism provided to convert the speed and torque provided by the magnetic field exposure of the magnet 41. In an embodiment, the gearbox 42 is of the type having input and output in a coaxial relation. The reduction mechanism may have a reduction ratio of transmission between the input and the output, i.e., the output (connected to the leadscrew 45) rotates slower than the input (connected to the magnet 41), though the contrary arrangement is possible. The gearbox 42 outputs the torque via its shaft 42B. The shaft 42B may be interfaced to the first tube 23 of the barrel member 20 by a bearing 43. The bearing 43 may be received and supported by the bearing support 23B in the first tube 23, if the bearing support 23B is present. As suggested above, other means may be provided to block the bearing 43 in a desired axial location along direction L, such as circlips, a shoulder and circlip, etc. The bearing 43 may for instance be a thrust bearing, though other types of bearings may be used as well.

Another bearing 44 may be used to support the magnet 41. The bearing 44 may be lodged in the end cap 24, as a possibility. The bearing 44 may be a radial bearing supporting a shaft portion of the housing 41B. Accordingly, the driving unit of the magnet 41 and the gearbox 42 may be held between the bearings 43 and 44 as in FIGS. 3 and 4 and/or in FIG. 6, to minimize any frictional loss in the rotational output from the magnet 41 through the magnetic field actuation. The bearings 43 and/or 44 may be rolling element bearings. This being said, other types of bearings could be used as well, such as plain bearings.

A leadscrew 45 (a.k.a., threaded shaft, threaded rod, screw, endless screw) is coupled to the driving unit via a coupling 46. The coupling 46 may be a flexible coupling, for example, and is coupled at one end to the shaft 42B of the gearbox 42 (if present) or is alternatively coupled directly to a shaft of the magnet 41 (i.e., on the magnet housing 41A). The embodiment of flexible coupling 46 is given as an example, as other embodiments are contemplated, including set screws, rigid sleeves, etc. The leadscrew 45 is threaded for complementary operative engagement with the internal threading on the nut portion 35A of the piston member 30. A rotation of the leadscrew 45, as driven by the driving unit in the barrel member 20, consequently results in a translation of the piston member 30 along distraction direction L, in a telescopic movement.

The internal fixator apparatus 10 may be used in both growing and mature long bones. Although the internal fixator apparatus 10 is configured to be used for paediatric distraction procedures due to its internal implanting capability and location relative to growth plates, the internal fixator apparatus 10 may also be used in other treatments. According to an embodiment, the greatest outer diameter of the barrel member 20 and of the piston member 30, excluding the interfaces 22 and 33, ranges from 12 mm to 20 mm, facilitating its internal use by its relatively small diametrical dimensions. For example, the internal fixator apparatus 10 may be used in a compressive set-up to treat non-unions, namely permanent failure of healing following a broken bone. The fixator actuator 50 is configured to perform the remote-controlled programmable procedure. The fixator actuator 50 may create a electromagnetic field system to accelerate bone regeneration. For example, as shown in FIG. 5, an internal fixator system as the internal fixator apparatus 10, illustrated by the magnet 41, and a rotating magnet(s) (e.g., permanent magnet(s), electromagnet(s)), for example shown as a cross 51 and rotating in a clockwise manner to induce a rotation of the magnet 41 by opposite polarities. More specifically, to cause expansion or contraction of the internal fixator apparatus 10, the cylindrical magnet 41 rotatingly encased in the barrel member 20 is activated by an external controller via the fixator actuator 50. In the controller, the cross 51 of magnets (e.g., electro magnets, permanent magnets) exposes alternatively positive and negative charges. When rotating, this magnetic arrangement moves in such a way that the magnet 41 inside the internal fixator apparatus 10 performs two full rotations every time the fixator actuator's magnets complete one full turn. For every rotation completed by the fixator actuator 50, the internal fixator apparatus 10 extends a given distance along direction L, such as 0.025 mm, resulting in a precise and controlled lengthening procedure.

To reduce the incidence of errors, a controller operating the rotation of the fixator actuator 50 may include a screen, a keypad or like user interfaces, which allows the user to input the desired distraction value directly into the system. The fixator actuator 50 may include a stepper motor to execute precisely the correct number of rotations. Furthermore, the controller is password-protected, reducing the potential for human error.

The proposed internal fixator apparatus 10 combines some principles of a telescopic intramedullary limb-lengthening nail and the geometry of locking plates when used as interfaces 22 and 32. The holes in the interfaces 22 and 32 along the length of the internal fixator apparatus 10 allow the use of locking screws, which may maintain a small distance between the internal fixator apparatus 10 and the bone F and improve the quality of the fixation. The extension or contraction of the internal fixator apparatus 10 is driven by the magnetically-actuated leadscrew 45, which engages the nut portion 35A in the moving half of the internal fixator apparatus 10, i.e., the piston member 30. When the leadscrew 45 is rotated, the telescopic parts move away from each other and linear extension is naturally achieved along direction L, whether for distraction or compression. The permanent magnet 41, configured for rotation by being rotatably supported, may be coupled to gearbox 42. The gearbox 42 may be tasked with converting rotations of the permanent magnet 41 into applied torque. The fixator actuator 50, placed on the outside of the patient's limb, controls the internal fixator apparatus 10 in achieving limb lengthening increments of a desired value. For example, the internal fixator apparatus 10 may be actuated to cause limb lengthening increments of 1 mm per day, or more, or less depending on the patient. The internal fixator apparatus 10 may be both distracted and compressed by changing the magnetic field, such that it may be used in multiple orthopedic applications including limb lengthening (distraction) and bone malunion corrections (compression). The internal fixator apparatus 10 may be scaled up or down depending on the patient.

While the above disclosure describes actuation via a passive permanent magnet 41 inside the internal fixator apparatus 10, it is contemplated to provide other driving units inside the internal fixator apparatus 10, including the hardware to operate a pulsed electromagnetic field treatment (PEMF) through active electromagnets located inside the internal fixator apparatus 10. The electromagnets could emit a low intensity magnetic field that could contribute to bone regeneration, in addition to allowing the expansion or contraction of the internal fixator apparatus 10. Another option would be to couple the internal fixator apparatus 10 hardware producing a low-intensity pulsed ultrasound (LIPUS), also to accelerate bone regeneration.

Referring to FIG. 6, exemplary dimensions are given. The dimensions may vary depending on different factors. However, the dimensions given are representative of an embodiment of the internal fixator apparatus 10. The dimensions are:

D1=2.0±0.4 mm

D2=7.9±1.6 mm

D3=5.9±1.2 mm

D4=7.0±1.4 mm

D5=20.0±4.0 mm

D6=27.9±5.9 mm

1. An internal fixator apparatus comprising:
a barrel member having a bone interface adapted to be anchored to a first part of a bone in an extramedullary connection,
a piston member having a bone interface adapted to be anchored to a first part of a bone, the piston member including a threaded nut portion, the barrel member and the piston member being operatively connected to concurrently form a joint whereby the barrel member and the piston member are displaceable at least in translation relative to one another, and
a fixator mechanism inside the barrel member and the piston member, the fixator mechanism comprising at least a leadscrew threadingly engaged with the threaded nut portion, and at least one magnet connected to the leadscrew to rotate concurrently therewith, the magnet being rotatingly received in the barrel member.

2. The internal fixator apparatus according to claim 1, wherein the magnet is a permanent magnet received in a housing.

3. The internal fixator apparatus according to claim 2, wherein the housing has shaft portions.

4. The internal fixator apparatus according to claim 3, wherein one of the shaft portions is rotatably connected to the barrel member by a bearing.

5. The internal fixator apparatus according to claim 4, wherein the bearing is supported by an end cap of the barrel member, the end cap plugging an end of a tube of the barrel member.

6. The internal fixator apparatus according to claim 3, wherein the housing is coupled to a remainder of the fixator mechanism by one of the shaft portions.

7. The internal fixator apparatus according to claim 1, wherein the fixator mechanism has a reduction mechanism reducing a speed of rotation from the magnet to the leadscrew.

8. The internal fixator apparatus according to claim 1, wherein the barrel member has a tube portion slidingly received in an annular gap of the piston member.

9. The internal fixator apparatus according to claim 8, wherein the barrel member has at least a first tube and a second tube connected to one another and concurrently defining an inner cavity of the barrel member, the tube portion slidingly received in the annular gap of the piston member being part of the second tube.

10. The internal fixator apparatus according to claim 9, wherein an anti-rotation coupling is defined between the tube portion and the piston member.

11. The internal fixator apparatus according to claim 9, wherein the first tube has an internal flange.

12. The internal fixator apparatus according to claim 11, wherein a bearing is supported by the internal flange, the bearing being rotatably connected to the fixator mechanism.

13. The internal fixator apparatus according to claim 9, further comprising a third tube in the barrel member, the first tube and the third tube forming another annular gap in which the second tube is received, the second tube projecting out of the other annular gap to define the tube portion cooperating with the piston member.

14. The internal fixator apparatus according to claim 13, wherein the third tube has an internal flange, a bearing being supported by the internal flange, the bearing being rotatably connected to the fixator mechanism.

15. The internal fixator apparatus according to claim 8, wherein the piston member has a first tube and a second tube connected to one another and concurrently defining an inner cavity of the piston member including the threaded nut portion, the first tube and a second tube of the piston member defining the annular gap of the piston member.

16. The internal fixator apparatus according to claim 1, wherein the fixator mechanism includes a flexible coupling between the leadscrew and a remainder of the fixator mechanism.

17. The internal fixator apparatus according to claim 1, wherein the barrel member has a tubular body with a diameter ranging between 12 and 20 mm.

18. The internal fixator apparatus according to claim 1, wherein the bone interface of the barrel member and/or of the piston member is a plate projecting laterally from a tubular body of the barrel member and/or of the piston member.

19. The internal fixator apparatus according to claim 18, wherein the piston member and the barrel member both have the plate as the bone interface.

20. A system comprising:
the internal fixator apparatus according to claim 1, and a fixator actuator including at least one rotating magnet.
